(19) United States
(12) Patent Application Publication
(10) Pub. No.: US 2021/0193262 A1
(43) Pub. Date: Jun. 24, 2021

(54) SYSTEM AND METHOD FOR PREDICTING ANTIMICROBIAL PHENOTYPES USING ACCESSORY GENOMES

(71) Applicant: KONINKLIJKE PHILIPS N.V., Eindhoven (NL)

(72) Inventor: Helen Cecile Van Aggelen, Berkeley, CA (US)

(21) Appl. No.: 17/063,746

(22) Filed: Oct. 6, 2020

Related U.S. Application Data
(60) Provisional application No. 62/953,266, filed on Dec. 24, 2019.

(51) Int. Cl. G16B 30/10

(52) U.S. Cl. CPC G16B 30/10

(57) ABSTRACT

A method for predicting a drug resistance phenotype of a microbe, comprising: (i) receiving sequencing information for the microbe, comprising at least a portion of the microbe's accessory genome; (ii) determining an accessory genome similarity metric between the accessory genome of the microbe and the accessory genome of one or more microbes in a dataset of previously characterized microbes, wherein each microbe in the dataset of previously characterized microbes is associated with drug resistance information; (iii) predicting, based on the determined accessory genome similarity metrics, a drug resistance of the microbe; and (iv) reporting the predicted drug resistance of the microbe.

### FIELD OF THE DISCLOSURE

The present disclosure is directed generally to methods and systems for predicting one or more antimicrobial phenotypes of a microbe using accessory genome similarity comparisons.

### BACKGROUND

Whole-genome sequencing (WGS) is an important tool for genomics research, and has numerous applications for discovery, diagnosis, and other methodologies. For example, WGS is increasingly being used in clinical settings to identify infectious outbreaks and accurately type pathogens of interest. These activities rely on genomic comparison metrics. Genomic comparison, however, is complicated by the intra-species variation seen in many pathogens. Some portions of the genome are highly variable and can carry genes that are not present in other strains, such as resistance or virulence genes. These portions of the genome are often referred to as the variable or accessory genome. Other portions of the genome are highly conserved and carry genes that are needed for survival. These portions of the genome are often referred to as the core genome.

Another benefit of WGS is genomics-based drug resistance prediction, which is increasingly becoming a viable alternative to experimental testing. Whereas experimental testing from a pure culture typically takes several days, rapid sequencing has the potential to provide quicker results. Although DNA sequencing only informs on gene presence and does not provide insight into the expressed genes, the resistance genes provide valuable information on the mechanisms of resistance. Moreover, DNA-based predictions typically display less variability than experimental testing. These advantages make it an attractive alternative to experimental testing to inform epidemiology and treatment decisions.

However, resistance prediction using WGS is often inadequate. Since resistance is often multifactorial involving multiple genes, machine learning (ML) models are typically trained to reproduce experimentally determined phenotypes, thereby predicting resistance phenotypes. The accuracy of these ML predictions varies per drug and species, but is often poor. For some species, such as S. aureus, the resistome is simple and ML models typically reach sufficient accuracy, but for species with more complex resistomes models for certain drugs are poor. Limited accuracy may be due to discrepancies between expression and genome, or features that are unaccounted for in the model, since models typically predict based on a predefined feature space. Another aspect of concern may be that ML models can be considered hard to interpret, especially when they are not based on gene presence/absence but on more general genomic features.

### SUMMARY OF THE DISCLOSURE

There is a continued need for methods and systems that more accurately predict the antimicrobial phenotype of a microbe based on DNA sequencing information.

The present disclosure is directed to inventive methods and systems for predicting the antimicrobial phenotype of a microbe. Various embodiments and implementations herein are directed to a system or method that receives sequencing information for a microbe, comprising at least a portion of the microbe's accessory genome. The system compares the accessory genome sequencing information, which can optionally be reduced to a feature representation, to a dataset of previously characterized microbes, each comprising accessory genome sequencing information and associated drug resistance information. Based on the characterized accessory genome similarity between the microbe and the previously characterized microbes, the system predicts a drug resistance of the microbe and reports that determination. A clinician may utilize the predicted drug response to determine and enact an infection treatment regimen.

Generally, in one aspect, a method for predicting a drug resistance phenotype of a microbe is provided. The method includes: (i) receiving sequencing information for the microbe, comprising at least a portion of the microbe's accessory genome; (ii) determining an accessory genome similarity metric between the accessory genome of the microbe and the accessory genome of one or more microbes in a dataset of previously characterized microbes, wherein each microbe in the dataset of previously characterized microbes is associated with drug resistance information; (iii) predicting, based on the determined accessory genome similarity metrics, a drug resistance of the microbe; and (v) reporting the predicted drug resistance of the microbe.

According to an embodiment, the method further includes generating the dataset of previously characterized microbes, comprising obtaining accessory genome sequencing information and drug resistance information for a plurality of previously characterized microbes. According to an embodiment, the method further includes generating, for each previously characterized microbe, a plurality of accessory genome k-mers from the associated obtained accessory genome sequencing information; and generating, using the plurality of accessory genome k-mers, a feature representation of the accessory genome of each of the previously characterized microbes, wherein the step of determining an accessory genome similarity metric between the microbe and one or more microbes in a dataset of previously characterized microbes comprises use of the generated feature representations.

According to an embodiment, the method further includes: generating a plurality of accessory genome k-mers from the accessory genome sequencing information for the microbe; and generating, using the plurality of accessory genome k-mers, a feature representation of the accessory genome of the microbe, where the step of determining an accessory genome similarity metric between the microbe and one or more microbes in the dataset of previously characterized microbes comprises use of the generated feature representation.

According to an embodiment, the accessory genome similarity metric for a comparison of two microbes is generated via the inproduct of the feature representation associated with those two microbes.

According to an embodiment, each of at least a plurality of microbes in the dataset of previously characterized microbes is further associated with microbe phenotype data.

According to an embodiment, the method further includes utilizing the predicted drug resistance of the microbe to determine an infection treatment regimen; and enacting the infection treatment regimen.

According to an embodiment, the predicted drug resistance of the microbe comprises a confidence of the predicted drug resistance.

According to an embodiment, the set of previously characterized microbes and associated information is obtained from a public sequence information database.

According to another aspect is a system for predicting a drug resistance phenotype of a microbe. The system includes: sequencing information for each of a plurality of previously characterized microbes, comprising at least a portion of the accessory genome; a processor configured to: (i) receive sequencing information for the microbe, comprising at least a portion of the microbe's accessory genome; (ii) determine an accessory genome similarity metric between the accessory genome of the microbe and the accessory genomes of one or more microbes in a dataset of previously characterized microbes, wherein each microbe in the dataset of previously characterized microbes is associated with drug resistance information; and (iii) predict, based on the determined accessory genome similarity metrics, a drug resistance of the microbe; and a user interface (640) configured to report the predicted drug resistance of the microbe.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for predicting a drug resistance phenotype of a microbe, in accordance with an embodiment.

FIG. 2 is a flowchart of a method for predicting a drug resistance phenotype of a microbe, in accordance with an embodiment.

FIG. 3 is a table of test results, in accordance with an embodiment.

FIG. 4 is a graph of resistance information, in accordance with an embodiment.

FIG. 5 is a graph showing the histogram of absolute errors on predictions, in accordance with an embodiment.

FIG. 6 is a schematic representation of a drug resistance phenotype prediction system, in accordance with an embodiment.

### DETAILED DESCRIPTION OF EMBODIMENTS

The present disclosure describes various embodiments of a system and method for predicting the drug response of a microbe. Applicant has recognized and appreciated that it would be beneficial to provide a method and system that can utilize accessory genome information to predict drug response. The system receives sequencing information for a microbe, comprising at least a portion of the microbe's accessory genome. The system compares the accessory genome sequencing information, which can optionally be reduced to a feature representation, to a dataset of previously characterized microbes, each comprising accessory genome sequencing information and associated drug resistance information. Based on the characterized accessory genome similarity between the microbe and the previously characterized microbes, the system predicts a drug resistance of the microbe and reports that determination, such as to a user, user interface, or other display or system. A clinician may utilize the predicted drug response to determine and enact an infection treatment regimen. According to an embodiment, the prediction takes advantage of the large amounts of data collected as part of routine microbiology practice, augmented with genomics (using a platform such as IntelliSpace Epidemiology) and provides clarity into how a prediction is made, and the typical variation on the prediction as a measure of the potential error.

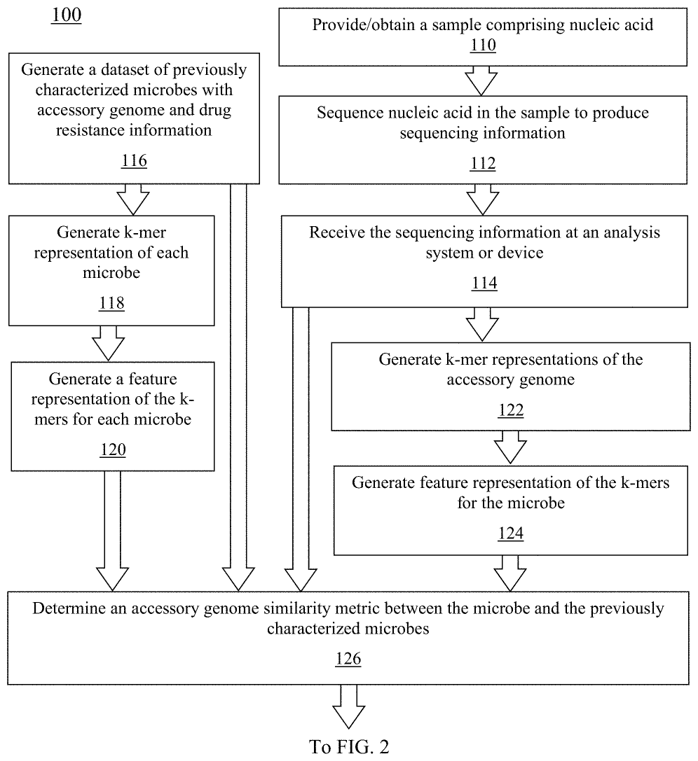

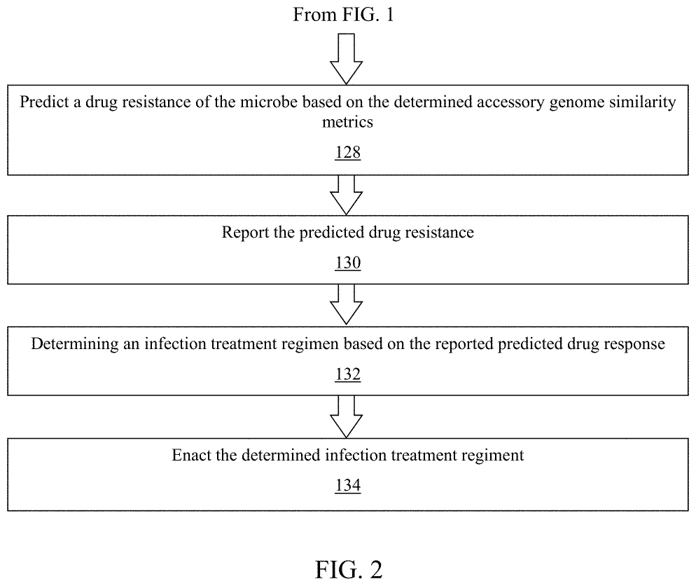

Referring to FIGS. 1 and 2, in one embodiment, is a flowchart of a method 100 for predicting a phenotype, such as a drug resistance, of an organism. At step 110 of the method, a sample comprising or potentially comprising nucleic acid to be sequenced is provided or received. The sample may comprise nucleic acid from one or more microorganisms such as bacteria, viruses, fungi, and/or from plants or animals, among many other sources. A sample may comprise nucleic acid molecules from one organism or from multiple organisms. Samples may be obtained in a clinical setting, from the environment, from indoor or outdoor surfaces, or from any other source. For example, samples may be obtained from a medical setting such as a hospital or other care or treatment facility, where one goal of analysis may be to examine the drug resistance of a sample. The sample can comprise one or multiple samples from the same location/hospital or pooled samples from multiple hospitals/locations. It is recognized that there is no limitation to the source of the sample, or the nucleic acid(s) in the sample.

The sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments. For some platforms, the nucleic acid may be fragmented using any method for nucleic acid fragmentation, such as shearing, sonication, enzymatic fragmentation, and/or chemical fragmentation, among other methods, and may be ligated to a sequencing adaptor or any other molecule or ligation partner.

At step 112 of the method, the sequencing platform sequences at least a portion of a nucleic acid from the sample, thereby generating sequencing information. The sequencing information is any information that represents the sequence of the nucleic acid being sequenced, where a genetic or genomic “sequence” is any series of one or more nucleic acid bases obtained by the sequencing platform. The sequencing platform can be any sequencing platform, including but not limited to any systems described or otherwise envisioned herein.

The sequencing information may be utilized immediately for additional steps of the methods described or otherwise envisioned herein, or may be stored for future use by this and other methods. Accordingly, the system may comprise or be in communication with local or remote data storage configured to store the sequencing information. The stored sequencing information may be in the form of waveforms, k-mers, and/or any other form of the sequencing information generated by the sequencing operation or the system.

At step 114 of the method, the system receives sequencing information from a sequencing operation. According to an embodiment, the sequencing information is communicated to or from the sequencing platform to a controller or other analysis module for downstream analysis and characterization. For example, according to one embodiment the sequencing platform may comprise a controller or other analysis module for downstream analysis and characterization. According to another embodiment, the sequencing platform communicates the generated sequencing information, in real-time or at certain time points, to a local or remote controller or other analysis module for downstream analysis and characterization. According to another embodiment, the system receives or retrieves the sequencing information from a database of stored sequencing signals.

The sequencing information generated or received by the system comprises nucleic acid sequence for the sample, such as for a microbe. The sequencing information includes coverage of at least a portion of the accessory genome of each genomic sample. The definition of the accessory genome may depend upon the identity of the genomic sample, but in general the accessory genome comprises regions of the genome that are highly variable between samples that are taxonomically, genealogically, and/or genomically related. For example, the accessory genome may comprise genes involved in resistance or virulence, among many other categories of genes. The accessory genome for an organism may be identified experimentally or may be defined in any other way.

At step 116 of the method, a dataset of characterized microbes is generated. The dataset comprises information about each of the characterized microbes, including but not limited to sequencing information for at least the accessory genome and phenotype information such as experimentally-derived drug resistance of the microbe. The information about each microbe may include any other phenotype information, gene expression information, and/or any other information. Once generated, the dataset can be stored in local or remote storage for future use, and/or can be utilized immediately in downstream steps of the method. The samples used for the dataset can be species-specific or universal, and different accessory genome regions may be used to predict different phenotypic traits (e.g. virulence factors versus resistance genes).

According to an embodiment, the dataset comprises a feature representation of the accessory genome of each characterized microbe, although many other methods of comparison between the dataset and a sample are possible. This enables a sample set-independent dataset, which make it possible to analyze new samples incrementally and compare them to older samples without having to re-compute results for older samples, which enables on-going and prospective analyses. However, other methods of comparison and analysis are possible.

To generate a sample set-independent feature representation of the accessory genome portion, the system can utilize feature representations of the accessory genomes of the organisms in the dataset. Accordingly, at optional step 118 of the method, the system generates a k-mer representation of at least the accessory genome of each organism in the dataset. According to an embodiment, the obtained accessory genome sequence information may represent one species or multiple species. The accessory genomes may be obtained from and/or represent any variety or diversity of species, strains, samples, or other metric. For example, according to one embodiment the plurality of accessory genomes may be obtained from a public and/or private database of genomes and/or accessory regions. Just one example of a possible database is the NCBI antimicrobial resistance database, although many other databases may be utilized. The NCBI antimicrobial resistance database provides sequence information about a wide variety of resistance genes for a wide variety of organisms, thus representing accessory genome information, and allows for download of the entire database. Associated with each resistance gene sequence is the identity of the organism from which the sequence was obtained.

The system then generates k-mers from the obtained accessory genome sequence information. The k-mers may be of any length, which can be predetermined or experimentally derived. The k-mers can be utilized immediately or can be stored for downstream use.

At optional step 120 of the method, the system generates a feature representation of the k-mers generated from the obtained accessory genome sequence information. The feature representation can be generated in a wide variety of different ways. According to one example, the k-mers are mapped to a sparse binary hash vector. According to another example, the k-mers are mapped to an embedded representation such as a dense floating point vector. The vector representation (hashing/embedding) may be species-specific or generic.

According to one embodiment, to map the k-mers to a sparse binary hash vector, a binary number representation for the k-mer is generated by mapping (A, T, C, G) onto 2-bit strings (00, 01, 10, 11) and concatenating them. The k-mers and their reverse complement can be treated as equal. The binary number is converted to its 10-base number, represented by i. The modulo is taken to convert i to a limited number of hashes. For example, I% 1,000,000,000. One benefit of this process is that the hashing scheme is generic and not tailored to the accessory genome k-mer space, such that the k-mer space of interest can be swapped out without changing the hashing. The vector representation of a sample, as described in an example below, will be a vector with 1's at every index for which the corresponding k-mer is present in the sample and 0 elsewhere.

According to one embodiment, to map the k-mers to an embedded representation, the resulting k-mers (1, 2, . . . x) are assigned to an index from 0 to x. Notably, the k-mers and their reverse complement can be treated as equal. The vector representation of a sample, as described in an example below, will be a vector with 1's at every index for which the corresponding k-mer is present in the sample and 0 elsewhere. This forms a one-hot representation of the x k-mer features.

One-hot representations can be generated for a set of y samples, and a matrix of y x x forms the input feature matrix for an embedding learning model. An autoencoder takes the y x x matrix as input and then passes it through fully connected layers of 1,000 and 500 and reconstructs it from this inner embedding of dimension 500. The model can be optimized to minimize the reconstruction error. The first layers of the model that compress the data to dimension 500 can then be used as embedding, such that any such input can be mapped onto a dense vector dimension 500, a very compact representation.

According to an embodiment, a k-mers from an organism may be considered present if it occurs more times than a given threshold, which may depend on the coverage and/or read number of the sample. Thus, the threshold may be adjusted by a user or automatically by the system depending on coverage and/or read information either provided to or determined by the system.

Once generated, the representation for each organism to be stored in or with the dataset can be stored in local or remote storage for future use, and/or can be utilized immediately in downstream steps of the method. For example, the vector representation of the accessory genome content can be stored in compact format (a sparse vector in the case of a hash vector or a small-dimensional dense vector in the case of an embedding representation) for future use, thus enabling quick and efficient comparison.

At optional step 122 of the method, the system generates k-mers from the obtained accessory genome sequence information for the sample microbe. The k-mers may be of any length, which can be predetermined or experimentally derived. The k-mers can be utilized immediately or can be stored for downstream use.

At optional step 124 of the method, the system generates a feature representation of the k-mers generated from the obtained accessory genome sequence information for the sample microbe. The feature representation can be generated in a wide variety of different ways. According to one example, the k-mers are mapped to a sparse binary hash vector. According to another example, the k-mers are mapped to an embedded representation such as a dense floating point vector. The vector representation (hashing/embedding) may be species-specific or generic.

Once generated, the representation for the microbe sample can be stored in local or remote storage for future use, and/or can be utilized immediately in downstream steps of the method.

At step 126 of the method, the system compares the accessory genome information of the microbe sample to the accessory genome information in the dataset to determine an accessory genome similarity metric between the microbe sample and the accessory genomes in the dataset. The accessory genome similarity metric can be calculated using any method for comparing the generated representations. According to an embodiment, the system computes a similarity metric between one or more pairs of samples via the inproduct of the vectors associated with the samples. Once generated, the similarity metric can be stored in local or remote storage for future use, and/or can be utilized immediately in downstream steps of the method.

At step 128 of the method, the system predicts the drug resistance phenotype of the sample microbe using the determined accessory genome similarity metrics between the microbe sample and the accessory genomes in the dataset. The prediction can be based on, for example, the phenotype of one or more of the characterized microbes in the dataset with an accessory genome that is most similar to the accessory genome of the sample microbe. For example, if the microbe or microbes in the dataset that most closely match the sample microbe are resistant to drug x, then the system may predict that the sample microbe is resistant to drug x as well. The function used to predict the phenotypic property from samples with high similarity can be an average, median, or more complex function of the measurements for the samples.

The prediction may comprise a threshold or any other method for improving the accuracy of the prediction. A threshold similarity may be required to ensure the sample microbe is sufficiently similar to the most similar microbes in the dataset, such that phenotype similarities are likely. A threshold finding of drug resistance or other phenotype may be required among the most similar microbes in the dataset, such that there is sufficient consistency among these most similar microbes to enable a confident prediction. The prediction may optionally comprise, for one or more similarity thresholds, a quantification of the variation, such as standard deviation, of the prediction. The threshold and quantification information can be provided as part of a report.

At step 130 of the method, the system provides the predicted drug response and/or other phenotype information of the microbe sample. The report may comprise any of the information described or otherwise envisioned herein. For example, the report may comprise the accessory genome similarity metrics between two or more accessory genomes, a list of one or more microbes in the dataset similar to the sample microbe, phenotype information about one or more microbes in the dataset, the predicted drug response and/or other predicted phenotype information, confidence or other quantification information, and/or any other information. The report may be electronic or printed, and may be stored. For example, the report may comprise a text-based file or other format. The report may be sortable or otherwise configured for organization to allow easy analysis and extraction of information.

According to an embodiment, the report or information may be stored in temporary and/or long-term memory or other storage. Additionally and/or alternatively, the report or information may be communicated or otherwise transmitted to another system, recipient, process, device, and/or other local or remote location.

According to an embodiment, once the report or information is generated, it can be provided to a researcher, clinician, or other user to review and implement an action or response based on the provided information. For example, a researcher, clinician or other user may utilize the information to determine one or more clinically actionable steps. Accordingly, at optional step 132, a researcher, clinician, or other individual utilizes the predicted phenotype of the microbe to determine a course of action, such as an infection treatment or prevention regimen. At step 134 of the method, the researcher, clinician, or other individual enacts the determined course of action.

Notably, the similarity calculation can be done in real time with a real-time sequencer such as Oxford Nanopore sequencers, among many other examples. The similarity calculation and prediction can be done at any point once the data available, and as more data is being sequenced the similarity metric will stabilize, indicating sufficiency of the sequencing data.

According to an embodiment, the method and systems described or otherwise envisioned herein can be used to flag phenotypes that are unexplained by the accessory genome features used. When experimental testing disagrees with a prediction, this may indicate that the sample has genomic features that are not considered in the calculation or may indicate an error in the experimental testing.

### EXAMPLE 1

The following non-limiting example describes embodiments of a method for generating a vector representation of an accessory genome. Although non-limiting, the example demonstrates a possible approach for generating the feature representation of the accessory genome according to the methods and systems described or otherwise envisioned herein. Many other approaches and variations are possible.

In this example, a set of k-mers of interest was defined by 31-merizing all sequences in NCBI's antimicrobial resistance database. The resulting 1,443,434 canonical 31-mers (k-mers and their reverse complement are treated as equal) were mapped onto a hash via the following function. A binary number representation for each k-mer was generated by mapping (A, T, C, G) onto 2-bit strings (00, 01, 10, 11) and concatenating them. The binary number was converted to its 10-base number, represented by i. The modulo was taken to convert i to a limited number of hashes. For example, i% 1,000,000,000. This mapped the 1,443,434 k-mers onto 1,427,603 unique hashes, such that there are not many hash collisions. The vector representation of a sample, as described in an example below, will be a vector with 1's at every index for which the corresponding k-mer is present in the sample and 0 elsewhere. The similarity between two samples i and j is calculated as:

$${S}_{ij} = \frac{{v}_{i} \cdot {v}_{j}}{‖ {v}_{i} ‖ ‖ {v}_{j} ‖} \qquad ( Eq . 1 )$$

where the ∥vᵢ∥ notation indicates the Euclidean vector norm.

### EXAMPLE 2

The following non-limiting example describes embodiments of a method for predicting a phenotype of an organism. Although non-limiting, the example demonstrates a possible approach for predicting a drug resistance phenotype of a sample organism using accessory genome information according to the methods and systems described or otherwise envisioned herein. Many other approaches and variations are possible.

In this example, a set of 269 E. faecium samples with whole genome sequencing data and associated drug resistance data in the form of minimum inhibitory concentrations (MIC) values (log 2-transformed) was obtained, and the accessory genome similarity for each sample was determined. From the set, 10% of the samples were randomly selected as the test set, yielding 27 test samples. The remaining 242 samples were used as training samples to represent the dataset of characterized microbes.

To give an idea of the feasibility of using similar samples for a drug resistance prediction, 25 out of the 27 test samples had at least one or more other samples in the dataset with similarity greater than 90%. This indicates that for a large fraction of samples, there are similar samples available in the database to rely on. On average, each sample in the test set has 55 similar samples in the training set (with >90% similarity) and the median number of similar samples is 55.

For each of the samples in the test set, all samples in the dataset with similarities over 0.9 were identified, and their associated MIC values (log 2-transformed) for all tested drugs was obtained. For each drug in the resulting set, the mean and standard deviation of the MIC values in the set were computed. Considering the mean as the predicted MIC value for each drug, the absolute error on the prediction relative to the known MIC value for the sample was quantified. This was done for each sample in the test set, such that there is a set of absolute errors for one or more drugs for each sample. Drugs for which there are no MIC values (in the test or training set) are ignored. To quantify the performance of this drug resistance prediction, the quality of the predictions made per sample was analyzed, as well as per drug and per individual prediction (per sample per drug).

Performance Per Sample in the Test Set

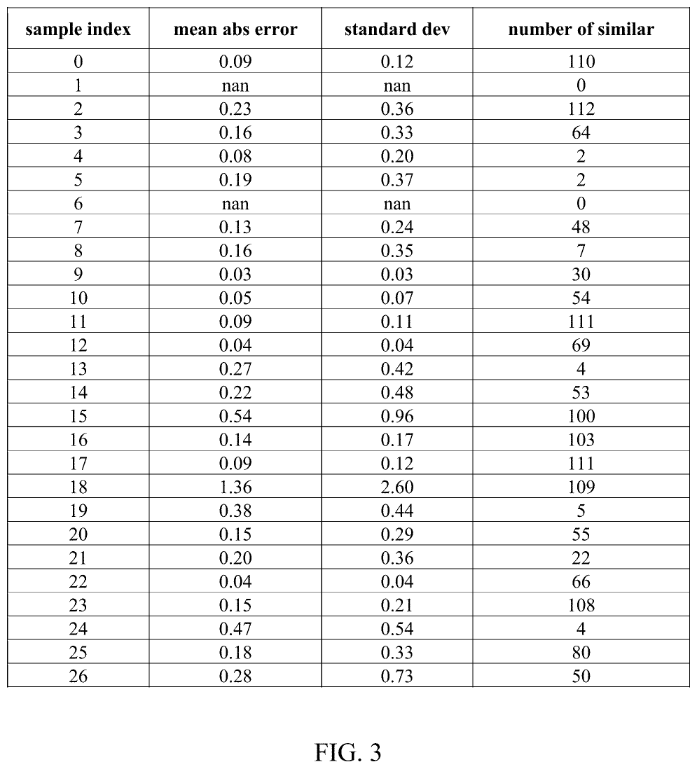

Referring to FIG. 3 is a table of results showing the mean absolute difference and standard deviation on the predictions made per sample in the test set. Samples 0 and 6 did not have any sufficiently similar samples in the dataset. For most samples, the mean absolute error is well below one. This is significant because MIC values within a 1-dilution factor difference are typically considered indistinguishable, as they are within the experimental error margin). Absolute errors smaller than one can thus be considered negligible, within the experimental error margin from the actual value.

Performance Per Drug in the Test Set

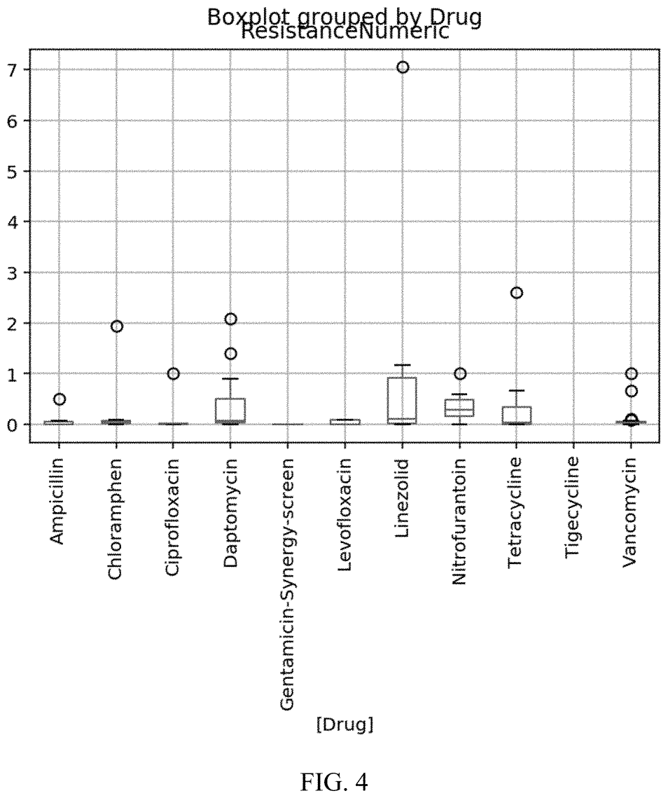

Referring to FIG. 4 is a graph of resistance information for 11 drugs. The graph shows the typical error on the MIC prediction per drug, displayed as boxplots over the whole test set. Outliers are indicated with circles. Note that all boxes (indicating the interquartile range, IQR) fall below one, and even most of the whiskers fall below one. Because of experimental error, differences in MIC dilution factors less than one are typically considered within the experimental error margin, so results within one dilution factor con be considered indistinguishable. For all drugs except Linezolid, typical errors (within 1.5*IQR from the median) in the prediction are below the experimental error margin. There are three outlier predictions with errors greater than two. For Tigecycline, there was not enough data to make a prediction, typically either the test or train sample was not tested for this drug.

Performance Per Drug Sample in the Test Set

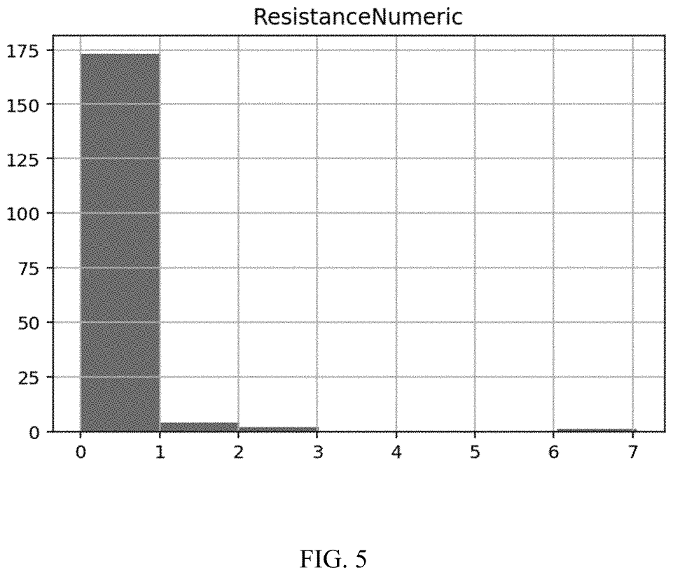

Referring to FIG. 5 is a histogram of the absolute errors on the predictions, considering each individual MIC prediction, where there were 180 predictions in total for the 27 samples. Out of the 180 predictions, 173 predictions (96%) were within one dilution factor accurate (absolute error <=1.0), and can be considered correct within the experimental error margin. Four drug predictions had errors greater than one but smaller than two (<=2.0) and only three predictions had errors greater than two. These results demonstrate very high accuracy for a model that is easily interpretable—a simple lookup of organisms with similar accessory genome features and aggregation of their experimental drug resistance features. As such, it functions differently from typical ML models, in which features are weighted in a manner that is hard to interpret. According to an embodiment, the method could thus be used alongside a ML model to provide an orthogonal prediction. Thus, according to an embodiment, the result of the prediction according to the method or system described or otherwise envisioned herein is compared to the prediction generated by a machine learning model. Similar results help confirm or reinforce the predictions, while disparate results may suggest an error or otherwise indicate uncertainty.

Although these examples are provided, many other approaches and embodiments are possible to accomplish the method and system described and otherwise envisioned herein.

The methods and systems described herein comprise several elements each comprising and analyzing millions of pieces of information. For example, generation of the feature representations corresponding to a dataset of accessory genomes large enough to function sufficiently, comprises the k-merization and feature representation generation using a set of sequencing data from hundreds or thousands of samples. Accordingly, the generated k-mers will number in the millions, and each must be analyzed to generate the feature representation or representations, thereby constituting millions of calculations. This is something the human mind is not equipped to perform, even with pen and pencil. Similarly, comparing the feature representation of one or more samples to the dataset representations comprises thousands or millions of comparisons/calculations. This is also something the human mind is not equipped to perform, even with pen and pencil.

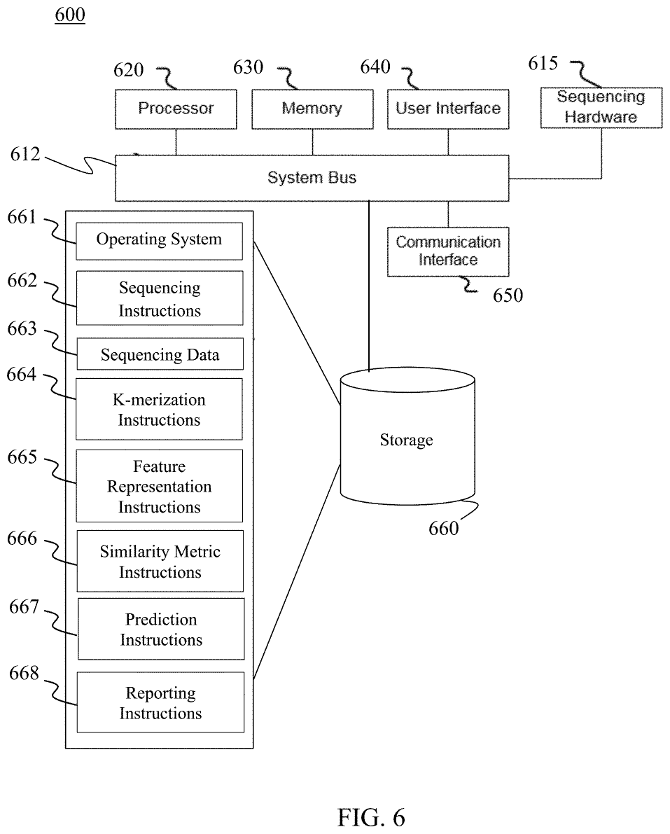

Referring to FIG. 6, in one embodiment, is a schematic representation of a system 600 for predicting a phenotype of a sample. System 600 may be any of the systems described or otherwise envisioned herein, and may comprise any of the components described or otherwise envisioned herein.

According to an embodiment, system 600 comprises one or more of a processor 620, memory 630, user interface 640, communications interface 650, and storage 660, interconnected via one or more system buses 612. In some embodiments, such as those where the system comprises or directly implements a sequencer or sequencing platform, the hardware may include additional sequencing hardware 615. It will be understood that FIG. 6 constitutes, in some respects, an abstraction and that the actual organization of the components of the system 600 may be different and more complex than illustrated.

According to an embodiment, system 600 comprises a processor 620 capable of executing instructions stored in memory 630 or storage 660 or otherwise processing data to, for example, perform one or more steps of the method. Processor 620 may be formed of one or multiple modules. Processor 620 may take any suitable form, including but not limited to a microprocessor, microcontroller, multiple microcontrollers, circuitry, field programmable gate array (FPGA), application-specific integrated circuit (ASIC), a single processor, or plural processors.

Memory 630 can take any suitable form, including a non-volatile memory and/or RAM. The memory 630 may include various memories such as, for example L1, L2, or L3 cache or system memory. As such, the memory 630 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 600. It will be apparent that, in embodiments where the processor implements one or more of the functions described herein in hardware, the software described as corresponding to such functionality in other embodiments may be omitted.

User interface 640 may include one or more devices for enabling communication with a user. The user interface can be any device or system that allows information to be conveyed and/or received, and may include a display, a mouse, and/or a keyboard for receiving user commands. In some embodiments, user interface 640 may include a command line interface or graphical user interface that may be presented to a remote terminal via communication interface 650. The user interface may be located with one or more other components of the system, or may located remote from the system and in communication via a wired and/or wireless communications network.

Communication interface 650 may include one or more devices for enabling communication with other hardware devices. For example, communication interface 650 may include a network interface card (NIC) configured to communicate according to the Ethernet protocol. Additionally, communication interface 650 may implement a TCP/IP stack for communication according to the TCP/IP protocols. Various alternative or additional hardware or configurations for communication interface 650 will be apparent.

Storage 660 may include one or more machine-readable storage media such as read-only memory (ROM), random-access memory (RAM), magnetic disk storage media, optical storage media, flash-memory devices, or similar storage media. In various embodiments, storage 660 may store instructions for execution by processor 620 or data upon which processor 620 may operate. For example, storage 660 may store an operating system 661 for controlling various operations of system 600. Where system 600 implements a sequencer and includes sequencing hardware 615, storage 660 may include sequencing instructions 662 for operating the sequencing hardware 615, and sequencing data 663 obtained by the sequencing hardware 615.

It will be apparent that various information described as stored in storage 660 may be additionally or alternatively stored in memory 630. In this respect, memory 630 may also be considered to constitute a storage device and storage 660 may be considered a memory. Various other arrangements will be apparent. Further, memory 630 and storage 660 may both be considered to be non-transitory machine-readable media. As used herein, the term non-transitory will be understood to exclude transitory signals but to include all forms of storage, including both volatile and non-volatile memories.

While system 600 is shown as including one of each described component, the various components may be duplicated in various embodiments. For example, processor 620 may include multiple microprocessors that are configured to independently execute the methods described herein or are configured to perform steps or subroutines of the methods described herein such that the multiple processors cooperate to achieve the functionality described herein. Further, where one or more components of system 600 is implemented in a cloud computing system, the various hardware components may belong to separate physical systems. For example, processor 620 may include a first processor in a first server and a second processor in a second server. Many other variations and configurations are possible.

According to an embodiment, storage 660 of system 600 may store one or more algorithms and/or instructions to carry out one or more functions or steps of the methods described or otherwise envisioned herein. For example, processor 620 may comprise one or more of sequencing instructions 662, k-merization instructions 664, feature representation instructions 665, similarity metric instructions 666, prediction instructions 667, and/or reporting instructions 678, among other instructions.

According to an embodiment, sequencing instructions 662 direct the system to operate a sequencing platform such as sequencing hardware 615. This may include any information necessary to process a sample and to generate and obtain sequencing data 663 from the sequencing platform. Sequencing instructions 662 may also instruct the system to communicate sequencing data 663 to another component of system 600. Additionally, sequencing instructions 662 may direct the system to store the sequencing data 663 in a local or remote database for retrieval and use by the system. The database may be located with system 600 or may be located remote from the system, such as in cloud storage and/or other remote storage.

According to an embodiment, k-merization instructions 664 direct the system to generate k-mers from sequence information. For example, as described or otherwise envisioned herein, the system may retrieve sequences from a database of gene sequences in order to generate a feature space for set-independent accessory genome analysis. These sequences may be gene sequences or genomic sequencing and must be converted to k-mers. The length of the generated k-mers can be determined by a user or can be experimentally derived. The k-merization instructions 664 may also direct the system to generate k-mers from sequence information for a sample that will be analyzed as described.

According to an embodiment, feature representation instructions 665 direct the system to generate a feature representation from k-mer information. For example, as described or otherwise envisioned herein, the system may retrieve sequences from a database of gene sequences in order to generate a feature space for set-independent accessory genome analysis. These sequences may be gene sequences or genomic sequencing and once they are converted to k-mers those k-mers are used to generate a feature representation. The representation can be any of those described or otherwise envisioned herein. Additionally, the feature representation instructions 665 also direct the system to generate a feature representation from the k-mers for the sample that will be analyzed as described.

According to an embodiment, similarity metric instructions 666 direct the system to determine a distance or similarly between two or more genomic samples. For example, the instructions inform the system to generate accessory genome similarity metrics by comparing the accessory genome of a sample to the accessory genomes of the dataset. The comparison may comprise feature representations of the accessory genomes.

According to an embodiment, prediction instructions 667 direct the system to predict, based on the accessory genome similarity metrics, the drug resistance phenotype of the sample microbe. The prediction can be based on, for example, the phenotype of one or more of the characterized microbes in the dataset with an accessory genome that is most similar to the accessory genome of the sample microbe. For example, if the microbe or microbes in the dataset that most closely match the sample microbe are resistant to drug x, then the system may predict that the sample microbe is resistant to drug x as well. The prediction may comprise a threshold, quantification, or any other method for improving the accuracy of the prediction.

According to an embodiment, reporting instructions 678 direct the system to generate, report, and/or provide the prediction to the user. This could be created in memory or a database, and/or displayed on a screen or other user interface such as via the user interface 640. The report may be a visual display, a printed text, an email, a transmission, and/or any other method of conveying information. The report may be provided locally or remotely, and thus the system or user interface may comprise or otherwise be connected to a communications system.

The genomic sample analysis system and method described or otherwise envisioned herein provides numerous advantages over existing systems. For example, the system improves the prediction of drug resistance compared to previous methods. According to certain embodiments, increased prediction of drug resistance has extremely beneficial impacts in a clinical setting. For example, this information can be important for monitoring infectious outbreaks and determination of pathogens of interest, as different infectious outbreaks may appear as related or unrelated with poor clustering results. It is also very important for quick and accurate treatment of infection. In a clinical setting in which an individual is fighting an infection, quickly and accurately identifying the microbe(s) participating in the infection, and the drug resistance profile of the microbe, can lead to faster and more accurate treatment. This can mean the difference between life and death in many settings and/or with many infections. Using the approach and/or system described or otherwise envisioned herein, a clinician or other healthcare provider can make significantly improved and more informed decisions and can better treat dangerous and often life-threatening infections.

According to an embodiment, the system also enables the creation of both sample set-independent comparisons for the accessory genome. Sample set-independent results make it possible to analyze new samples incrementally and compare them to older samples without having to re-compute results for older samples, which enables on-going and prospective analyses. This significantly improves the efficiency and speed of the system.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for predicting a drug resistance phenotype of a microbe, comprising:
receiving sequencing information for the microbe, comprising at least a portion of the microbe's accessory genome;
determining an accessory genome similarity metric between the accessory genome of the microbe and the accessory genome of one or more microbes in a dataset of previously characterized microbes, wherein each microbe in the dataset of previously characterized microbes is associated with drug resistance information;
predicting, based on the determined accessory genome similarity metrics, a drug resistance of the microbe; and
reporting the predicted drug resistance of the microbe.

2. The method of claim 1, further comprising the step of generating the dataset of previously characterized microbes, comprising obtaining accessory genome sequencing information and drug resistance information for a plurality of previously characterized microbes.

3. The method of claim 2, further comprising the steps:
generating, for each previously characterized microbe, a plurality of accessory genome k-mers from the associated obtained accessory genome sequencing information; and
generating, using the plurality of accessory genome k-mers, a feature representation of the accessory genome of each of the previously characterized microbes;
wherein the step of determining an accessory genome similarity metric between the microbe and one or more microbes in a dataset of previously characterized microbes comprises use of the generated feature representations.

4. The method of claim 3, further comprising:
generating a plurality of accessory genome k-mers from the accessory genome sequencing information for the microbe; and
generating, using the plurality of accessory genome k-mers, a feature representation of the accessory genome of the microbe;
wherein the step of determining an accessory genome similarity metric between the microbe and one or more microbes in the dataset of previously characterized microbes comprises use of the generated feature representation.

5. The method of claim 4, wherein the accessory genome similarity metric for a comparison of two microbes is generated via the inproduct of the feature representation associated with those two microbes.

6. The method of claim 1, wherein each of at least a plurality of microbes in the dataset of previously characterized microbes is further associated with microbe phenotype data.

7. The method of claim 1, further comprising the steps:
utilizing the predicted drug resistance of the microbe to determine an infection treatment regimen; and
enacting the infection treatment regimen.

8. The method of claim 1, wherein the predicted drug resistance of the microbe comprises a confidence of the predicted drug resistance.

9. The method of claim 1, wherein the set of previously characterized microbes and associated information is obtained from a public sequence information database.

10. A system for predicting a drug resistance phenotype of a microbe, comprising:
sequencing information for each of a plurality of previously characterized microbes, comprising at least a portion of the accessory genome;
a processor configured to: (i) receive sequencing information for the microbe, comprising at least a portion of the microbe's accessory genome; (ii) determine an accessory genome similarity metric between the microbe and one or more microbes in a dataset of previously characterized microbes, wherein each microbe in the dataset of previously characterized microbes is associated with drug resistance information; and (iii) predict, based on the determined accessory genome similarity metrics, a drug resistance of the microbe; and
a user interface configured to report the predicted drug resistance of the microbe.

11. The system of claim 10, wherein the processor is further configured to generate the dataset of previously characterized microbes, comprising obtaining accessory genome sequencing information and drug resistance information for a plurality of previously characterized microbes.

12. The system of claim 11, wherein the processor is further configured to: generate, for each previously characterized microbe, a plurality of accessory genome k-mers from the associated obtained accessory genome sequencing information; and generate, using the plurality of accessory genome k-mers, a feature representation of the accessory genome of each of the previously characterized microbes, wherein determining an accessory genome similarity metric between the microbe and one or more microbes in a dataset of previously characterized microbes comprises use of the generated feature representations.

13. The system of claim 12, wherein the processor is further configured to: generate a plurality of accessory genome k-mers from the accessory genome sequencing information for the microbe; and generate, using the plurality of accessory genome k-mers, a feature representation of the accessory genome of the microbe, where determining an accessory genome similarity metric between the microbe and one or more microbes in the dataset of previously characterized microbes comprises use of the generated feature representation.

14. The system of claim 13, wherein the accessory genome similarity metric for a comparison of two microbes is generated via the inproduct of the feature representation associated with those two microbes.

15. The system of claim 10, wherein the predicted drug resistance of the microbe comprises a confidence of the predicted drug resistance.
